(19) United States
(12) Patent Application Publication
(10) Pub. No.: US 2017/0319385 A1
(43) Pub. Date: Nov. 9, 2017

(54) EYE DROP DISPENSING DEVICE

(71) Applicant: Ocular Technology Inc., Virginia Beach, VA (US)

(72) Inventors: Bernard Fresco, Toronto (CA); Bruce Gitelman, Toronto (CA); David Snaith, Toronto (CA); Steve Copeland, Barrie (CA)

(21) Appl. No.: 15/586,268

(22) Filed: May 3, 2017

Related U.S. Application Data
(60) Provisional application No. 62/331,074, filed on May 3, 2016.

(51) Int. Cl. A61F 9/00

(52) U.S. Cl. CPC A61F 9/0026; A61M 2210/0618; A61M 2210/0612; A61M 2205/586; A61M 2209/088; A61M 2207/00; A61M 2210/0606

(57) ABSTRACT

In an aspect, an eye drop delivery device is provided and includes a lower periorbital registration portion that is positionable on a recipient's face and an upper bottle alignment portion that is arranged in spaced-apart relationship from the lower periorbital registration portion. The upper bottle alignment portion is configured to receive an eye drop medicament bottle. The upper bottle alignment portion optionally includes a first upper arm and a second upper arm arranged in spaced-apart relationship to permit the placement of the eye drop medicament bottle therebetween. The first and second upper arms define a slot configured to receive the neck ring portion of an eye drop medicament bottle. The slot being sized to complement industry-standard eye drop medicament bottle neck sizing.

### CROSS-REFERENCE TO RELATED APPLICATIONS

This application claims the benefit of and priority to U.S. Provisional Patent Application No. 62/331,074 filed May 3, 2016, the disclosure of which is incorporated herein by reference in its entirety.

### FIELD

The present disclosure relates to the field of eye drop delivery devices, and in particular to an eye drop delivery device that retains and positions a medicament bottle at a preferred location for eye drop delivery.

### BACKGROUND

In general the administration of eye drops from an eye drop bottle to a recipient's eyes is unpleasant. The experience is generally uncomfortable, and there is a strong reflexive action for the recipient to move or otherwise avoid having the drop impact upon the eye surface. This can create issues with medicament delivery, where proper placement of the drop is difficult, and where inaccuracies can arise in the required dosage to be delivered.

It is also sometimes difficult to ensure that the tip of the eye drop bottle dues not inadvertently contact the eyes or eyelashes of the person receiving the eye drops. This can lead to contamination of the medicament solution which can facilitate the persistence of an infection or can facilitate the transfer of an infection from one person to another person.

It would be desirable to provide an eye drop delivery device that permits for greater control over drop delivery at the desired location.

### SUMMARY

According to an aspect of the disclosure, provided is an eye drop delivery device. The eye drop delivery device comprises a lower periorbital registration portion that is positionable on a recipient's face, and an upper bottle alignment portion that is arranged in spaced-apart relationship from the lower periorbital registration portion. The upper bottle alignment portion being configured to receive an eye drop medicament bottle.

### BRIEF DESCRIPTION OF THE DRAWINGS

The foregoing and other features and advantages will be apparent from the following description of the disclosure as illustrated in the accompanying drawings. The accompanying drawings, which are incorporated herein and form a part of the specification, further serve to explain the principles of the disclosure and to enable a person skilled in the pertinent art to make and use the disclosure. The drawings are not to scale.

FIG. 1 is a perspective view of an eye drop delivery device according to an embodiment of the present disclosure.

FIG. 2 is a top view of the eye drop delivery device according to FIG. 1

FIG. 3 is a side view of the eye drop delivery device according to FIG. 1.

FIG. 4 is a side view of the eye drop delivery device according to FIG. 1, showing an eye drop bottle mounted in the dispensing position.

FIG. 5 is a side view of the eye drop delivery device according to FIG. 1, shown in relation to a recipient's face.

FIG. 6 is a side view of the eye drop delivery device according to FIG. 1, showing an eye drop bottle mounted in a position suitable for being packaged.

FIG. 7 is a side view of the eye drop delivery device according to FIG. 1, showing the mounting of an eye drop bottle upon the eye drop delivery device.

FIG. 8 shows another way of mounting the eye drop delivery device on an eye drop bottle in a packaging position.

### DETAILED DESCRIPTION

Specific embodiments of the present disclosure are now described with reference to the figures, wherein like reference numbers indicate identical or functionally similar elements. The following detailed description is merely exemplary in nature and is not intended to limit the disclosure or the application and uses of the disclosure. Furthermore, there is no intention to be bound by any expressed or implied theory presented in the preceding technical field, background, brief summary or the following detailed description.

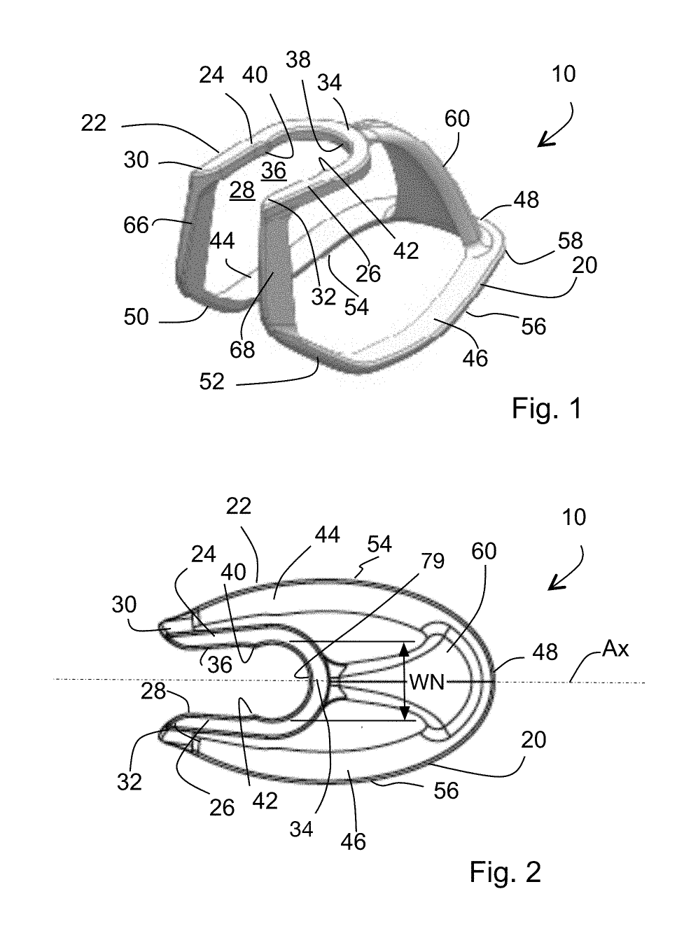

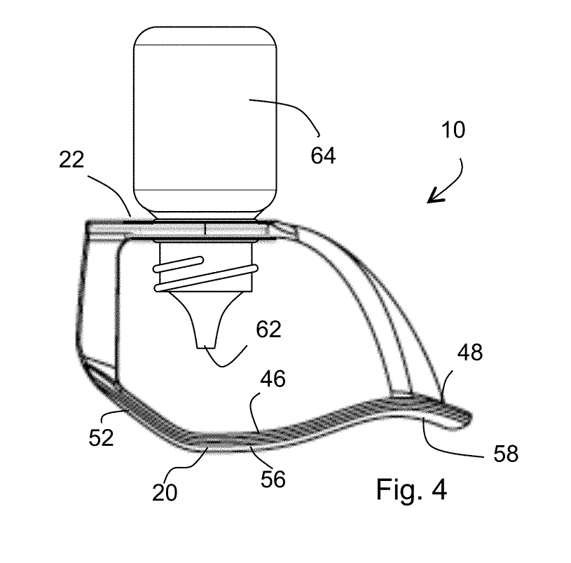

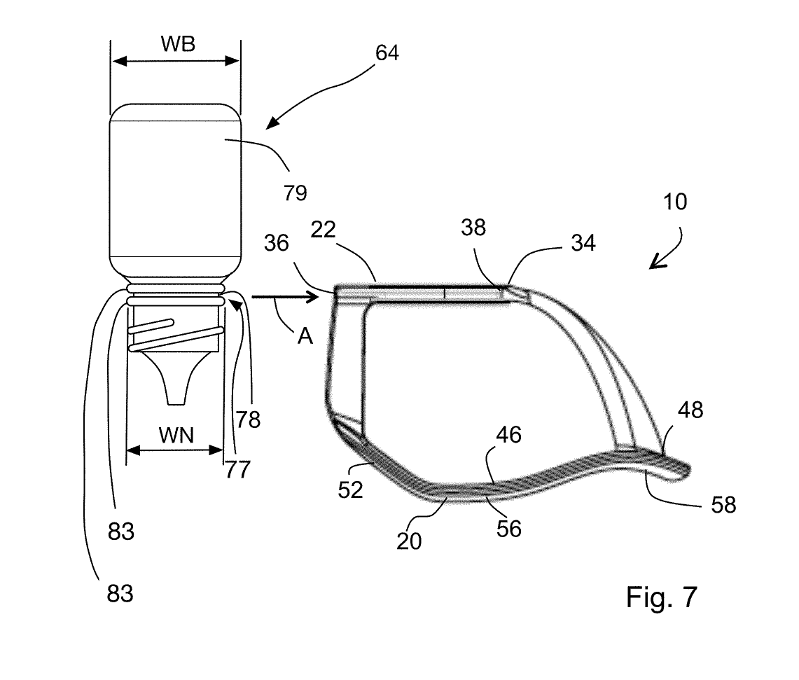

Referring now to FIGS. 1 to 3, shown is an eye drop delivery device 10 (which may also be referred to simply as a delivery device 10). The delivery device 10 includes a lower periorbital registration portion (herein referred to as lower section 20) and an upper bottle alignment portion (herein referred to as upper section 22). The upper section 22 is generally planar and yoke-shaped, and is configured to receive an eye drop medicament bottle (shown in FIGS. 4-7 at 64). The eye drop medicament bottle 64 may, for readability, also be referred as an eye drop bottle 64, or simply as a bottle 64. The section 22 includes a first upper arm 24 and a second upper arm 26 arranged to define an opening 28 between a first end 30 and a second end 32, and an upper bridge 34 opposite the opening 28 where the first and second upper arms 24, 26 interconnect. The first and second upper arms 24, 26 are arranged in spaced-apart relationship to permit the placement of an eye drop medicament bottle therebetween. The first and second upper arms 24, 26 define a slot 36 that extends from the opening 28 towards the upper bridge 34, the slot 36 terminating at a radiused wall 38 (also, more broadly referred to as a bottle holding region 38) dimensioned to generally complement industry-standard bottle neck sizing. Put another way, the slot 36 may be sized to be larger (i.e. wider) than the neck (shown at 77 in FIG. 7) of the eye drop bottle (shown at 64 in FIG. 7), while also be less wide than the body of the eye drop bottle 64. The body of the eye drop bottle 64 is shown at 79. For example, the neck 77 may have a width WN that is about 13 mm and a body 79 having a width WB that is about 24 mm. In such embodiments, the bottle holding region 38 of the slot 36 has a width WS that is greater than about 13 mm and less than about 24 mm, so as to permit the neck 77 to fit therein, while preventing the body 79 from passing therethrough. While the example eye drop bottle 64 had dimensions WN and WB of about 13 mm and about 24 mm, respectively, it is possible for the bottle 64 to have had other dimensions for WN and WB, such as about 13 mm and about 38 mm. In such an example, the bottle holding region 38 of the slot 36 may have a width WS that is greater than about 13 mm and less than about 38 mm.

While the width WS of the bottle holding region 38 may be anywhere between the widths WN and WB (i.e. any value that is greater than the width WN and less than the width WB), it is advantageous for the width WS of the bottle holding region 38 to be larger than the width WN of the neck 77 by less than about 2 mm, to help locate the tip 62 of the bottle 64 in a predictable position relative to the eye drop dispensing device 10. Thus, the width WS of the bottle holding region 38 may be between about 13 mm and about 15 mm. Even more advantageously, the width WS of the bottle holding region 38 may be larger than the width WN of the neck 77 by less than about 1 mm to help locate the tip 62 more precisely. Thus, the width WS of the bottle holding region 38 may be between about 13 mm and about 14 mm.

The slot 36 may also include a pair of opposing projections 40, 42 which serve to releasably retain the eye drop bottle 64 within the bottle holding region 38. The projections 40 and 42 have a pass-through width that may be slightly smaller than (e.g. by about 1-2 millimetres) the width WS of the bottle holding region 38.

The lower section 20 is similarly yoke-shaped, and is positionable on a recipient's face. The lower section 20 includes a first lower arm 44 and a second lower arm 46 arranged in spaced-apart relationship, the first and second lower arms 44, 46 being interconnected at a lower bridge 48. The lower section 20 is generally contoured to follow a predefined anatomical profile in the periorbital region of the eye. Accordingly, the lower section 20, in particular the first and second lower arms 44, 46 define a plurality of registration portions positioning the dispensing device 10 upon the recipient. As shown, the first and second lower arms 44, 46 include respective nasal registration portions 50, 52, as well as respective central registration portions 54, 56. The lower bridge 48 of the first and second lower arms 44, 46 define a lateral registration portion 58.

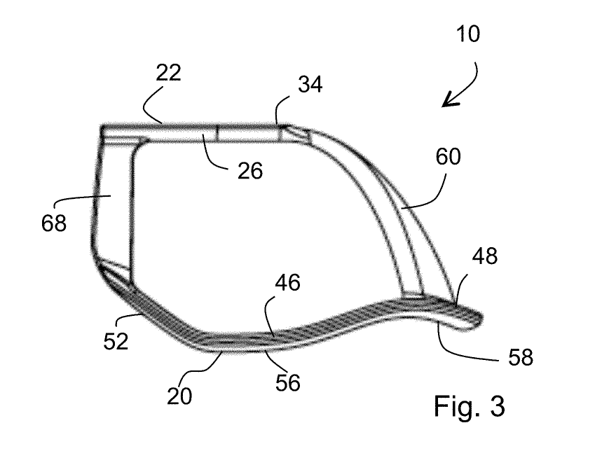

As seen in FIG. 3, the upper section 22 and the lower section 20 are arranged in spaced apart relationship, and are interconnected by at least one support 60 extending between the lower bridge 48 of the lower section 20 and the upper bridge 34 of the upper section 22. The distance between the upper section 22 and the lower section 20 is such that upon mounting the eye drop bottle in the dispensing position as shown in FIG. 4, the tip of the eye drop bottle is situated sufficiently distant from the lower section 20, thereby reducing the likelihood of contact between the tip 62 (also referred as the dispensing tip 62) of the eye drop bottle 64 and the recipient's eye or surrounding area.

The delivery device 10 may additionally include one or more connector segments between the upper section 22 and the lower section 20. As shown, the delivery device 10 is provided with a first connector segment 66 extending from the first end 30 of the upper section 22 to the first lower arm 44 of the lower section 20. Similarly, a second connector segment 68 is provided that extends from the second end 32 of the upper section 22 to the second lower arm 46 of the lower section 20.

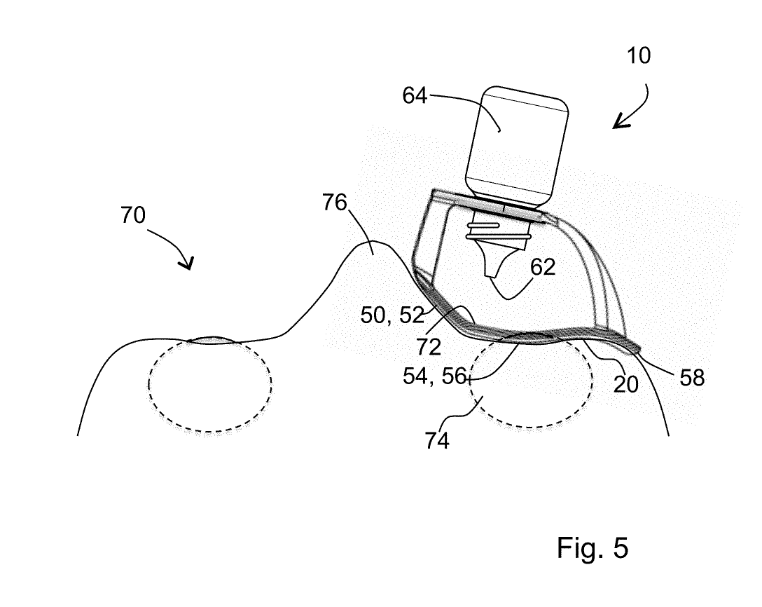

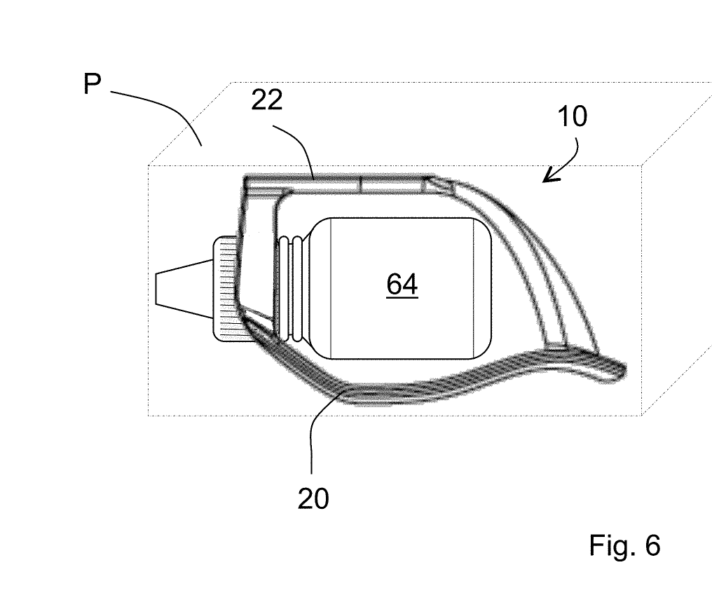

As shown in FIG. 5, the delivery device 10 is positionable on the face 70 of the recipient to support the eye drop bottle 64, in particular the tip 62 above the inner corners, shown at 72 of the eyes 74. The delivery device 10, in particular the lower section 20 registers against the periorbital region of the recipient at multiple points to promote accurate and reproducible alignment and delivery of the medicament. As shown, the nasal registration portions 50, 52 engage and register against the side of the recipient's nose 76. The central registration portions 54, 56 engage the periorbital region around the eye, that is against at least one of the upper or lower surfaces around the eye-socket. The lateral registration portion 58 may engage the region of the recipient's face laterally outward of the eye socket. It will be appreciated that while the figures show complete engagement of each of the registration portions with the recipient's face, in use, it is preferable that at least 2 of the noted registration portions engage features of the recipient's face.

The upper section 22, and lower section 20, together with the support 60 and first and second connector segments 66, 68 collectively form a one-piece structure. For instance these components may be formed using any suitable molding process, including but not limited to injection molding. As shown, the dispensing device 10 has been designed to be devoid of undercuts or other features that would otherwise impede the direct removal of the molded product from the molding environment. As undercuts generally require a complex arrangement of mold slides or displaceable cores, the overall cost of manufacture can quickly escalate. Instead, with the absence of undercuts in the dispensing device 10, manufacturing costs associated with complex mold tooling can be minimized. Although the design is well suited for one-piece manufacturing, in some embodiments, the delivery device may be formed from discrete interconnected components.

The delivery device 10 may be wholly or partly made of a substantially rigid material such as a rigid polymer. As a non-limiting example, a suitable rigid polymer for the delivery device would be polypropylene. Suitable materials may also include those which resist microbial contamination, and permit for cleaning via dishwasher and/or sterilization techniques. Where injection molded, the delivery device is suitable for manufacture without the use of mold slides.

The delivery device 10 may be provided in a range of sizes to suite a range of anatomical profiles, generally in relation to age of the recipient. In other embodiments, the delivery device 10 may be custom made to suit the anatomical profile of a particular recipient, for example by constructing the delivery device 10 based on three-dimensional (3-D) topographical scans taken of the recipient's facial anatomy. Delivery devices personalized in this way may be formed using additive manufacturing techniques, such as 3-D printing.

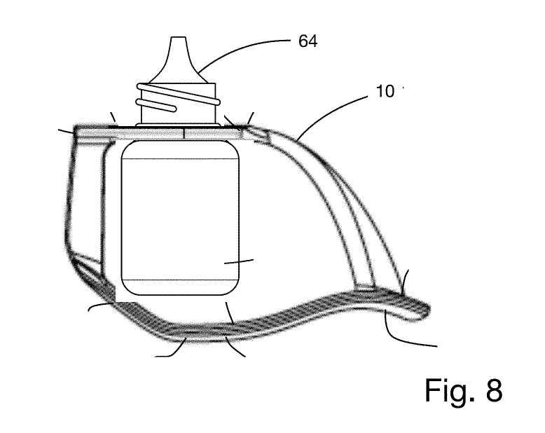

The delivery device 10 may be provided in kit form with the required medicament provided in the eye drop bottle. To permit for effective and compact packaging, the delivery device may be mounted on the eye drop bottle 64 in a packaged position, as shown in FIG. 6. The delivery device 10 provides sufficient clearance between the upper section 22 and the lower section 20 to receive the eye drop bottle 64. The first and second connector segments 66, 68 (as best seen in FIG. 1) are arranged to provide sufficient clearance therebetween to receive the neck portion of the eye drop bottle 64. In this way, the combined delivery device 10 and eye drop bottle 64 maybe compactly stored in a suitable packaging P, as shown. In the example shown the packaging P is a box, however any other suitable packaging may be used. FIG. 8 shows another packaging position for the eye drop delivery device 10 and the bottle 64.

To assemble, the neck (shown at 77) of the eye drop bottle 64 is aligned to the slot 36 provided between the first upper arm 24 and the second upper arm 26 (see FIG. 7). In the example shown, a region 78 of the neck 77 that is between two neck rings shown at 83 is aligned to the slot 36 provided between the first upper arm 24 and the second upper arm 26. The eye drop bottle 64 is moved in the direction of arrow A along the slot 36 towards the radiused wall 38 at the upper bridge 34. It will be appreciated that while the delivery device is formed of a substantially rigid material, its polymeric construction will afford sufficient flex to permit the region 78 between the neck rings 83 (or more generally, the neck 77) of the eye drop bottle 64 to slide past the pair of opposing projections 40, 42, thereby promoting a secure but removable mounting of the eye drop bottle 64 upon the delivery device 10. As the delivery device 10 provides largely open access to the cap 80 of the eye drop bottle 64, the cap 80 can be removed from the bottle 64 after the mounting of the bottle on the delivery device 10. This further reduces the likelihood of contamination by inadvertently touching the tip 62 during handling of the eye drop bottle 64.

Once the eye drop bottle 64 is securely mounted on the delivery device 10, the recipient tips their head back (e.g. by lying down) so that their face 70 faces upwards. The delivery device 10 is placed on the face 70 of the recipient in the periorbital area. In the embodiment shown, this placement would involve engaging the lower section 20 upon the at least 2 registration features on the recipient's face. While the delivery device 10 is being positioned on the face 70 of the recipient, the eyes 74 of the recipient may be closed. Thus, the delivery device 10 may be positioned on the face 70 of the recipient by another person. After the delivery device is suitably positioned, the eye drop bottle 64 is squeezed to release a select number of eye drops. The delivery device 10 may be removed after the eye drops have been administered, and the recipient can open the treated eye 74. Opening the eye 74 permits the eye drops that have accumulated at the inner corner 72 to enter the eye 74. It will be noted that such a technique permits the recipient to receive eye drops comfortably, without the need for the eye drops to be dropped into the recipient's eyes from some height onto the recipient's cornea, as has been done in the past in an attempt to avoid contact of the eye drop bottle with the recipient's eye.

It will be appreciated that while the eye drop bottle 64 is designed to position against the radiused wall 38 of the upper section 22, the configuration of the slot 36 between the first and second upper arms 24, 26 permits for a range of positions, for example if it were necessary to bring the tip 62 of the eye drop bottle 64 further laterally inwards (towards the nose). While a single pair of projections 40, 42 are provided in the embodiment shown, in other embodiments, additional pairs of projections may be provided to provide additional set positions of the eye drop bottle 64 upon the delivery device 10.

Having regard to the figures, it will be appreciated that the delivery device 10 is symmetrical through axis Ax (see FIG. 2). Accordingly, the delivery device 10 may be used to deliver eye drops to both the left and right eye, as required.

While various embodiments have been described above, it should be understood that they have been presented only as illustrations and examples of the present disclosure, and not by way of limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. An eye drop delivery device, comprising:
a lower periorbital registration portion that is positionable on a recipient's face; and
an upper bottle alignment portion that is arranged in spaced-apart relationship from the lower periorbital registration portion, the upper bottle alignment portion being configured to receive an eye drop medicament bottle.

2. The eye drop delivery device according to claim 1, wherein the upper bottle alignment portion includes a first upper arm and a second upper arm arranged in spaced-apart relationship to permit the placement of the eye drop medicament bottle therebetween.

3. The eye drop delivery device according to claim 2, wherein the first and second upper arms define a slot configured to receive a neck of an eye drop medicament bottle, the slot having a bottle holding region that is sized to receive the neck of the eye drop medicament bottle, and to prevent entry of a body of the eye drop medicament bottle.

4. The eye drop delivery device according to claim 3, wherein the bottle holding region has a width that is less than about 2 mm larger than a width of the neck of the eye drop medicament bottle.

5. The eye drop delivery device according to claim 3, wherein the bottle holding region has a width that is between about 13 mm and about 15 mm.

6. The eye drop delivery device according to claim 1, wherein the lower periorbital registration portion includes a first lower arm and a second lower arm arranged in spaced-apart relationship, the first and second lower arms defining a plurality of registration portions shaped for positioning the eye drop delivery device on the recipient.

7. The eye drop delivery device according to claim 6, wherein each of the first and second lower arms define a nasal registration portion for registration against the side of the recipient's nose, and a central registration portion for registration against at least one of the upper or lower surfaces around the recipient's eye socket.

8. The eye drop delivery device according to claim 7, wherein the first and second lower arms additionally define a lateral registration portion to engage the region of the recipient's face laterally outward from the eye socket.

9. The eye drop delivery device according to claim 6, wherein at least two registration portions are used to position the eye drop delivery device upon the recipient's face.

10. The eye drop delivery device according to claim 1, wherein the eye drop delivery device is formed as a one-piece structure.

11. The eye drop delivery device according to claim 1, wherein the eye drop delivery device is of a design devoid of undercut.

12. The eye drop delivery device according to claim 1, wherein the eye drop delivery device is injection molded.

13. The eye drop delivery device according to claim 1, wherein the eye drop delivery device is formed through an additive manufacturing process based on a personalized 3-D topographical scan of the recipient's facial anatomy.

14. The eye drop delivery device according to claim 1, wherein the eye drop delivery device is configured to receive the eye drop medicament bottle in a plurality of positions.

15. The eye drop delivery device according to claim 14, wherein the eye drop medicament bottle is mountable upon the eye drop delivery device in a first packaged position, and wherein the eye drop medicament bottle is mountable upon the eye drop delivery device in a second dispensing position.

16. The eye drop delivery device according to claim 3, wherein the eye drop medicament bottle is laterally positionable along the slot to provide adjustability in the dispensing of the medicament solution.
